(19) United States
(12) Patent Application Publication
(10) Pub. No.: US 2004/0226852 A1
(43) Pub. Date: Nov. 18, 2004

(54) Method of stabilizing azithromycin

(76) Inventors: Michael Pesachovich, Givat-Shmuel (IL); Sarah Isaacs, Hertzelia (IL); Claude Singer, Kfar Saba (IL); Eduard Schwartz, Rehovot (IL); Edit Berger, Tel -Aviv (IL)

Correspondence Address: KENYON & KENYON, ONE BROADWAY, NEW YORK, NY, 10004, US

(21) Appl. No.: 10/782,047

(22) Filed: Feb. 19, 2004

Related U.S. Application Data
(60) Provisional application No. 60/448,946, filed on Feb. 19, 2003.

(51) Int. Cl. B65D 1/09; B65D 83/04; B65D 85/42; A61K 31/7052

(52) U.S. Cl. 206/528; 514/28

(57) ABSTRACT

A method of packaging of azithromycin which provides improved stability of azithromycin upon storage.

### RELATED APPLICATIONS

This application claims the benefit of U.S. Provisional Patent Application 60/448,946 filed Feb. 19, 2003 which is incorporated herein by reference.

### FIELD OF THE INVENTION

The invention encompasses methods of packaging azithromycin to prevent the degradation of azithromycin upon storage.

### BACKGROUND OF THE INVENTION

Azithromycin has the chemical name [2R-(2R*,3S*,4R*,5R*,8R*,10R*,11R*,12S*,13S*,14R*)]-13-[(2,6-dideoxy-3-C-methyl-3-O-methyl-α-L-ribo-hexopyranosyl)oxy]-2-ethyl-3,4,10-trihydroxy-3,5,6,8,10,12,14-heptamethyl-11-[[3,4,6-trideoxy-3-(dimethylamino)-β-D-xylo-hexopyranosyl]oxy]-1-oxa-6-azacyclopentadecan-15-one and the following chemical structure:

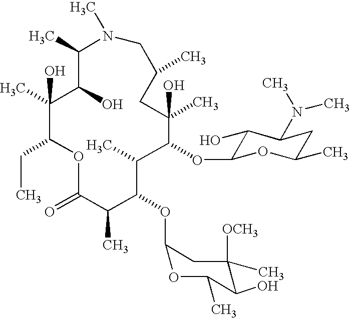

Azithromycin is one of the macrolide antibiotics, so named because they contain a many-membered lactone ring to which are attached one or more deoxy sugars. Other macrolide antibiotics include erythromycin and clarithromycin. Azithromycin and the other macrolide antibiotics are bacteriostatic agents which act by binding to the 50S ribosomal subunit of susceptible microorganisms, and thus interfering with microbial protein synthesis.

Macrolide antibiotics of the erythromycin class, such as erythromycin A, are known to be unstable in an acidic environment and are inactivated by gastric acids. See, Goodman and Gilman's, The Pharmacological Basis of Therapeutics 1137 (Joel G. Hardman et al., eds.) 9th ed. 1996; C. Vinckier et al., Int. J. Pharmaceutics, 55, 67-76 (1989); T. Cachet et al., Int. J. Pharmaceutics, 55, 59-65 (1989); E. F. Fiese and S. H. Steffen, J. Antimicrobial Chemother., 25 (suppl.A) 39-47 (1990).

Azithromycin is a semi-synthetic antibiotic which differs chemically from erythromycin in that a methyl-substituted nitrogen atom is incorporated into the lactone ring. The replacement of the keto group in the lactone ring with the N-methyl group in the lactone ring improves the stability of azithromycin over erythromycin in an acidic environment.

U.S. Pat. Nos. 4,517,359 and 4,474,768 disclose processes for the preparation of azithromycin and the use of azithromycin as an antibiotic. These patents are incorporated herein by reference.

Azithromycin is subject to degradation that may occur during manufacture and/or storage. For example, azithromycin is susceptible to degradation if exposed to elevated temperatures and/or air during manufacturing processes, processes that include formulation of the pharmaceutical dosage form. One particular example of oxidative degradation is the oxidation of the exocyclic amine group of azithromycin. The azithromycin susceptibility to degradation leads to deviation of the drug product from regulatory purity requirements even prior to the product reaching the patient. In addition, once formulated, azithromycin tends to degrade under normal storage conditions, which may result in the presence of unacceptable levels of impurities at the time of administration.

Therefore, a continuing need exists to provide consistent dosages of arithromycin by providing methods that delay or prevent the production of degradation products by improving storage methods for azithromycin.

### SUMMARY OF THE INVENTION

The invention encompasses methods for packaging azithromycin which shows improved stability of azithromycin upon storage. In particular, the present invention encompasses methods for packaging azithromycin comprising storing azithromycin in a gas impermeable package made of at least one sheet of gas impermeable material, wherein after storage azithromycin degradation products do not exceed 5%, preferably less than about 3% by weight of azithromycin. The gas impermeable material is impermeable to oxidizing agents, preferably to oxygen. The gas impermeable package may be selected from any material known in the art. The sheet may be a laminated sheet preferably an aluminum laminate package. The package may be comprised of a bag or a pouch.

Another embodiment of the invention encompasses methods for storing azithromycin comprising storing azithromycin in a gas impermeable package comprising at least one layer, wherein the intimate layer is prepared from a gas impermeable material and is capable of being sealed. The gas impermeable material may be selected from any material known in the art. The gas impermeable material is preferably an aluminum laminate. After the storage azithromycin degradation products do not exceed 5%, preferably less than about 3% by weight of the azithromycin. In another embodiment, the azithromycin storage conditions include at least one of a temperature of about 25° C. to about 55° C.; 60% relative humidity; or a time of at least one month.

Another embodiment of the invention encompasses methods for packaging azithromycin comprising storing a unit dosage of azithromycin in a gas impermeable package. The gas impermeable package may be selected from any material known in the art. The gas impermeable package is preferably an aluminum laminate package.

Another embodiment of the invention encompasses methods for packaging azithromycin wherein less than about 5% of azithromycin monohydrate is transformed to the dihydrate form on storage for one year.

The degradation products may be identified by HPLC relative retention times of about 0.26, 0.34, 0.37, and 0.80.

### BRIEF DESCRIPTION OF THE FIGURES

FIG. 1 illustrates the X-ray powder diffraction pattern for azithromycin Form A.

FIG. 2 illustrates the X-ray powder diffraction pattern for the dihydrate.

### DETAILED DESCRIPTION OF THE INVENTION

Definitions

The term “azithromycin” includes solvates and hydrates thereof, e.g. propanol solvate, ethanol solvate, monohydrate and other crystalline forms.

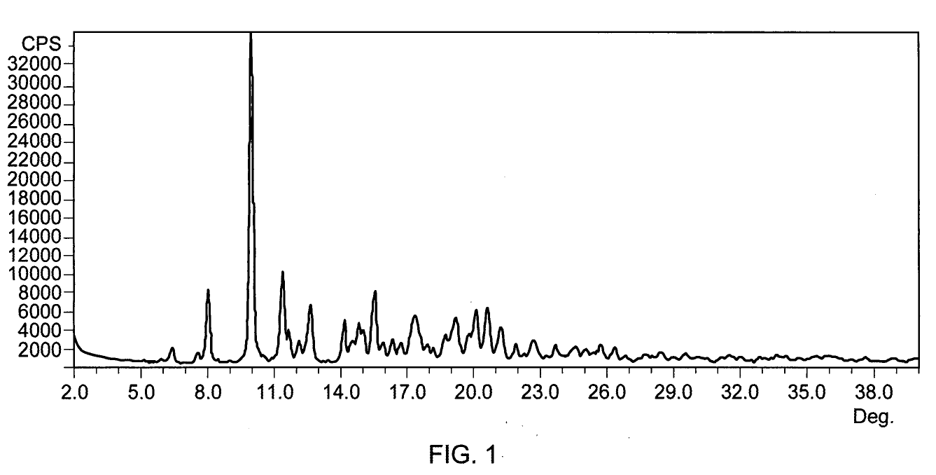

The term “Form A” refers to a crystalline form of azithromycin having an X-ray powder diffraction with peaks at 6.3, 8.0, 10.0, 11.4, 11.6, 12.0, 12.6, 14.0, 14.5, 14.7, 15.0, 15.4, 15.9, 17.3, 18.7, 19.1, 20.0, 20.3, and 21.2 degrees two-theta. The peaks of Form A are listed in FIG. 1.

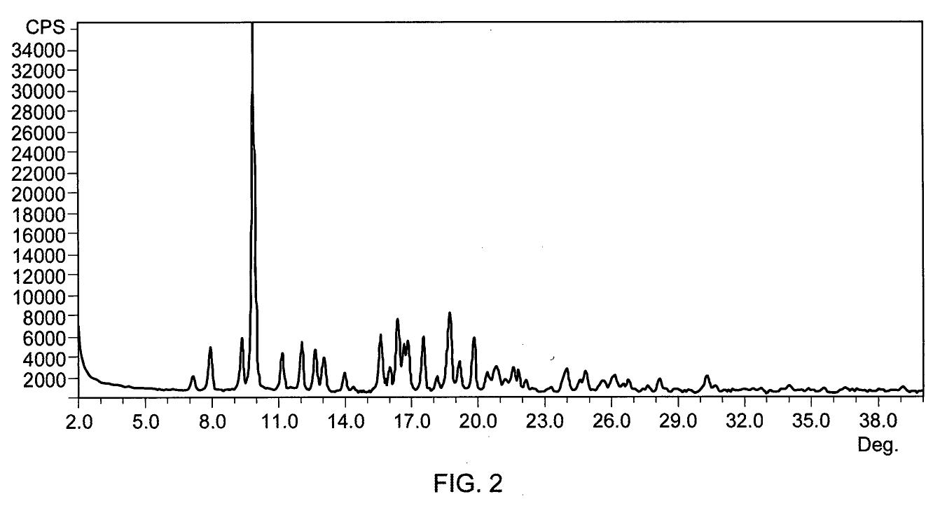

The term “dihydrate azithromycin” refers to a crystalline form of azithromycin having an X-ray powder diffraction with peaks at 9.3, 12.1, 13.0, 16.4, and 18.7 degrees two-theta. The peaks of the dihydrate are listed in FIG. 2.

As used herein, the term “AZT” refers to azithromycin. As used herein, the term “DMAZT” refers to azaerythromycin A (USP), desmethyl azithromycin. The term “API” refers to active pharmaceutical ingredient. The term “intimate layer” refers to the layer of gas impermeable packaging which contacts the stored material.

As used herein, the term “gas impermeable” refers to a property of a material wherein the passage of gases through the material is delayed or prohibited. As used with packaging, “gas impermeable” refers to the packaging of products having improved barrier characteristics better than those of low density polyethylene (LDPE) having been manufactured by coextrusion, lamination, metallization, or coating.

As used herein, the term “unit dosage form” refers to the amount of azithromycin, or a derivative thereof, which is effective to produce a therapeutic effect in a subject.

As used herein, the term “lamination” refers to a situation when two or more individuals films are bonded together with special adhesives and run through rolling, heated cylinders to produce a composite film structure.

### DESCRIPTION OF THE INVENTION

Azithromycin is unstable and prone to produce degradation products upon manufacture and/or storage. Not to be bound by theory, it is believed that one degradation pathway is the oxidation of azithromycin in the presence of oxidizing agents, such as oxygen. The degradation products may be identified by HPLC relative retention times of about 0.26, 0.34, 0.37, and 0.80.

Thus, the invention encompasses methods of storing azithromycin and containers for storing azithromycin comprising at least one gas impermeable material wherein the containers diminish or protect azithromycin from either: a) degradation, in particular degradation by oxidation, or b) changing of azithromycin solvate composition (water or solvent or a combination thereof as compared to the composition before AZT is packaged).

The advantage of using at least one gas impermeable container to protect azithromycin from oxidation is the increase in azithromycin shelf life.

Also, the invention encompasses containers for storing azithromycin comprising at least one gas impermeable material effective to protect azithromycin from degradation, especially at elevated temperatures.

One embodiment of the invention encompasses containers for storing azithromycin comprising a container having at least one gas impermeable material and capable of being sealed. Generally, the container may include bottles, jar, pouches, envelopes, bags, and the like. Preferably, the container is in the form of a pouch or bag and comprises at least one gas impermeable material in the form of a sheet. The gas impermeable package may be selected from any material known in the art to be gas impermeable. Preferably, the material is oxygen and/or air impermeable. Preferably, the material is in the form of at least one laminate aluminum containing polymer. More preferably, the material is in the form of laminate aluminum containing polymer. An example of the polymer is polyethylene. The sheet may contact itself to form an envelope or a bag or may contact a second sheet of gas impermeable material to form a cavity wherein the azithromycin is placed.

There may be a better stabilizing effect of proposed double aluminum laminate instead of polyethylene in aluminum laminate.

Another embodiment of the invention encompasses methods for storing azithromycin comprising placing azithromycin in a container comprising at least one gas impermeable layer having an exterior and an intimate layer, wherein the intimate layer is prepared from a gas impermeable material and is capable of being sealed. The azithromycin may be in the form of a unit dosage of azithromycin. The unit dosage form may be a 250 mg, 500 mg, or 600 mg unit.

Another embodiment of the invention encompasses methods for packaging azithromycin, wherein the packaging delays or prevents azithromycin from degradation caused by water, oxygen, or both. As used herein, the term “delay or prevents degradation” as applied to azithromycin refers to the formation of no more than 5% by weight of azithromycin degradation products, preferably, no more than 3% by weight of degradation products. In another embodiment, the azithromycin storage conditions include at least one of a temperature of about 25° C. to about 55° C.; 60% relative humidity; or a time of at least one month. Alternatively, the packaging allows for less than about 5% of azithromycin monohydrate to transform to azithromycin dihydrate upon storage for one year. In another embodiment, the azithromycin storage conditions include at least one of a temperature of about 25° C. to about 55° C.; wherein at 55° C. with uncontrolled humidity the azithromycin monohydrate is stable for at least one month, preferable for at least 3 months, and wherein at 25° C. with 60% relative humidity, the azithromycin monohydrate is stable for at least one month, preferable at least 3 months and more preferably for at least one year.

The regular packaging material, which is used for stability studies, is polyethylene of low density wrapped into aluminum laminate. The polyethylene of low density is penetrable for gases.

The stability of azithromycin is substantially increased when the material is packed directly in aluminum laminate bags. Use of this packaging material enables to store safely the azithromycin at normal temperatures.

Having described the invention with reference to certain preferred embodiments, other embodiments will become apparent to one skilled in the art from consideration of the specification. The invention is further defined by reference to the following examples describing in detail the identification, isolation, and purification methods of the invention. It will be apparent to those skilled in the art that many modifications, both to materials and methods, may be practiced without departing from the scope of the invention.

### EXAMPLES

Although the following examples illustrate the practice of the present invention in some of its embodiments, the examples should not be construed as limiting the scope of the invention. Other embodiments will be apparent to one skilled in the art from consideration of the specification and examples.

### Example 1

Several azithromycin samples were analyzed using HPLC to determine the level of impurities within each sample. The analytical conditions of the HPLC were column of 150×4.6 mm; packing material of Kromasil KR 100-5C18, 5μ; and an eluent of 40% 0.05 M K₂HPO₄ adjusted to a pH of 8.2 and 60% acetonitrile. The flow rate was 0.9 ml/min; the detector set at 210 nm; and column temperature of 30° C. The samples were injected into the HPLC and run for over 35 min. The impurities were determined by their relative retention times (RRT) as compared to azithromycin and were reported as a weight percent (versus azithromycin) of the total composition. Additional impurities found in the samples were reported under “other RRT” as a weight percent of the azithromycin content. The results of the analytical tests is summarized in Table 1. Table 1 demonstrates a finding of the main azithromycin degradation products where azithromycin batches have been stored under uncontrolled temperature conditions (25° C. and higher) in regular packages (intimate package is LDPE and exterior is aluminum laminate). The lowest row of the table sums up each impurity content for all batches. The raw data reveals that the main degradants of azithromycin upon storage are RRT 0.26, 0.34, 0.37, and 0.80.

| | RRT (%) | | | | | | | | | | | Other | |
|---|---|---|---|---|---|---|---|---|---|---|---|---|---|
| AZT Batch | 0.16 | 0.18 | 0.23 | 0.26 | 0.34 | 0.37 | 0.40 | 0.49 | 0.60 | 0.80 | 0.88 | RRT % | Total % |
| Batch 1 | ND | <0.1 | ND | 0.13 | 0.45 | 0.14 | ND | ND | ND | 0.25 | ND | 0.45 | 1.7 |
| Batch 2 | ND | <0.1 | ND | ND | 0.32 | <0.1 | ND | ND | ND | 0.24 | ND | 0.49 | 1.3 |
| Batch 3 | 0.15 | ND | ND | 0.16 | 0.64 | 0.32 | ND | ND | ND | <0.1 | ND | 0.64 | 1.5 |
| Batch 4 | ND | ND | ND | <0.1 | <0.1 | <0.1 | ND | <0.1 | ND | <0.1 | ND | 0.00 | 0.0 |
| Batch 5 | ND | ND | ND | <0.1 | <0.1 | <0.1 | ND | <0.1 | ND | 0.11 | ND | 0.11 | 0.2 |
| Batch 6 | ND | <0.1 | ND | <0.1 | ND | <0.1 | <0.1 | <0.1 | ND | ND | ND | 0.00 | 0.0 |
| Batch 7 | ND | <0.1 | ND | <0.1 | <0.1 | <0.1 | ND | ND | ND | ND | ND | 0.16 | 0.2 |
| Batch 7 | ND | <0.1 | ND | 0.41 | 0.37 | 0.23 | 0.22 | ND | ND | 0.20 | ND | 0.41 | 1.4 |
| Batch 8 | ND | <0.1 | ND | 0.14 | 0.16 | <0.1 | ND | ND | ND | ND | ND | 0.16 | 0.4 |
| Batch 8 | ND | <0.1 | ND | 0.28 | 0.28 | 0.19 | 0.21 | ND | ND | 0.14 | ND | 0.28 | 1.2 |
| Batch 9 | ND | ND | ND | ND | <0.1 | <0.1 | ND | ND | ND | ND | ND | 0.00 | 0.0 |
| Batch 9 | ND | ND | ND | 0.29 | 0.40 | 0.17 | ND | <0.1 | ND | 0.12 | ND | 0.40 | 1.2 |
| Batch 10 | ND | ND | <0.1 | ND | 0.13 | <0.1 | ND | ND | ND | <0.1 | ND | 0.13 | 0.2 |
| Batch 10 | ND | ND | ND | <0.1 | 0.18 | 0.11 | ND | 0.10 | ND | <0.1 | ND | 0.18 | 0.5 |
| Batch 11 | ND | ND | ND | <0.1 | 0.13 | <0.1 | ND | <0.1 | ND | <0.1 | ND | 0.16 | 0.4 |
| Batch 12 | <0.1 | ND | <0.1 | 0.18 | 0.23 | <0.1 | <0.1 | ND | ND | <0.1 | ND | 0.23 | 0.5 |
| Sum of | 0.15 | 0.00 | 0.00 | 1.59 | 3.29 | 1.16 | 0.43 | 0.10 | 0.00 | 1.06 | 0.00 | | |
| impurities | | | | | | | | | | | | | |

### Examples 2

### Storage Testing

Three samples of azithromycin were separately packaged in a standard polyethylene bag, and then the polyethylene bags containing azithromycin were separately packaged into aluminum bags with silica gel. The stored azithromycin was submitted to stability programs either long term or accelerated to determine the effect upon azithromycin stability and the production of degradation products. The longer term stability program comprised submitting the sample to a temperature of about 25° C.±2° C. at a relative humidity of 60%±5%. The accelerated program comprised submitting the sample to a temperature of about 40° C.±2° C. at a relative humidity of 75%±5%. The samples were analyzed at regular intervals to determine the impurity profiles as assayed by HPLC using the technique described in Example 1. The water content was determined by Karl Fischer methodology; and the ethanol content was determined by gas chromatography. The results of these tests are summarized in Table 2, where “Any %” means any kind of impurity that gives the highest content in azithromycin.

TABLE 2
Azithromycin Stability in polyethylene bag

| AZT | Time | | Impurities | | % | % |
|---|---|---|---|---|---|---|
| Batch | (months) | Temp. (° C.) | Any % | Total % | Water | Ethanol |
| Batch | 0 | | 0.12 | 0.33 | 2.99 | 2.2 |
| No. 4 | 3^{a} | 25° C. | 0.55 | 2.14 | 2.97 | 2.2 |
| | 1^{b} | 40° C. | 0.45 | 1.93 | 3.13 | 2.1 |
| | 2^{b} | | 0.65 | 3.10 | 2.65 | 1.8 |
| | 3^{b} | | 0.77 | 3.71 | 2.95 | 1.8 |
| Batch | 0 | | 0.12 | 0.22 | 3.83 | 1.9 |
| No. 5 | 3^{a} | 25° C. | 0.49 | 2.17 | 2.93 | 1.8 |
| | 1^{b} | 40° C. | 0.43 | 1.77 | 3.22 | 1.8 |
| | 2^{b} | | 0.72 | 2.78 | 2.86 | 1.6 |
| | 3^{b} | | 1.11 | 5.07 | 3.27 | 1.5 |
| Batch | 0 | | <0.1% | <0.1% | 3.78 | 2.0 |
| No. 6 | 3^{a} | 25° C. | 0.32 | 1.40 | 2.75 | 2.0 |
| | 1^{b} | 40° C. | 0.44 | 1.71 | 3.21 | 1.9 |
| | 2^{b} | | 0.62 | 2.08 | 2.80 | 1.9 |
| | 3^{b} | | 0.81 | 3.94 | 3.12 | 1.7 |

^{a}Long term program.
^{b}Accelerated program.

Evaluation of results shown in Table 2 demonstrated that more degradation products were produced at higher temperatures, i.e. 40° C., as compared to either the starting material or at lower temperatures, i.e. 25° C. Table 3 contains a detailed presentation of the impurity profile for the tested batches wherein the impurities were reported as by RRT and weight percentage of the total composition.

TABLE 3
Extended Analytical Profile for Azithromycin

| | Time | | Impurities RRT (%) | | | | |
|---|---|---|---|---|---|---|---|
| AZT Batch | (months) | Temp° C. | 0.26 | 0.35 | 0.38 | 0.40 | 0.82 |
| Batch No. 4 | 0 | | <0.1 | 0.12 | <0.1 | <0.1 | |
| | 3^{a} | 25 | 0.40 | 0.43 | 0.29 | 0.21 | 0.34 |
| | 1^{b} | 40 | 0.45 | 0.42 | 0.28 | 0.22 | 0.31 |
| | 2^{b} | | 0.65 | 0.61 | 0.50 | 0.22 | 0.46 |
| | 3^{b} | | 0.72 | 0.77 | 0.50 | 0.37 | 0.61 |
| | 3^{b} | 55 | 0.78 | 0.91 | 0.61 | 0.34 | 0.73 |
| Batch No. 5 | 0 | | <0.1 | <0.1 | <0.1 | <0.1 | 0.12 |
| | 3^{a} | 25 | 0.49 | 0.46 | 0.44 | 0.15 | 0.18 |
| | 1^{b} | 40 | 0.39 | 0.43 | 0.23 | 0.25 | 0.25 |
| | 2^{b} | | 0.59 | 0.72 | 0.37 | 0.19 | 0.35 |
| | 3^{b} | | 1.41 | 0.76 | 0.72 | 0.19 | 0.52 |
| | 3^{b} | 55 | 1.27 | 1.19 | 1.22 | 0.06 | 0.91 |
| Batch No. 6 | 0 | | <0.1 | <0.1 | <0.1 | <0.1 | <0.1 |
| | 3^{a} | 25 | 0.31 | 0.32 | 0.3 | 0.1 | 0.12 |
| | 1^{b} | 40 | 0.44 | 0.40 | 0.26 | 0.25 | <0.1 |
| | 2^{b} | | 0.49 | 0.62 | 0.27 | 0.16 | 0.20 |
| | 3^{b} | | 0.74 | 0.71 | 0.67 | 0.19 | 0.47 |
| | 3^{b} | 55 | 0.92 | 0.87 | 0.92 | 0.06 | 0.65 |

^{a}Long term program.
^{b}Accelerated program.

### Example 3

### Azithromycin Stability as a Function of Storage Temperature

Samples of azithromycin were placed in storage bags and each batch sample was analyzed after storage at a variety of temperatures using the analytical techniques as described in Example 1. Each batch was packaged in a polyethylene bag and subsequently, each bag was packaged in an aluminum bag with silica gel. Table 4 summarizes the effects of storage temperature on the production of azithromycin degradation products. The results demonstrate that storing azithromycin at low temperatures (+5° C.) leads to inhibition of the production of degradation products.

TABLE 4
Azithromycin Stability as a Function of Storage Temperature

| AZT | Time | | RRT (%) | | | | Other | |
|---|---|---|---|---|---|---|---|---|
| Batch | (months) | T° C. | 0.26 | 0.34 | 0.37 | 0.80 | RRT % | Total % |
| Batch | 0 | | <0.1 | 0.07 | 0.03 | <0.1 | <0.1 | 0.1 |
| No. 4 | 3 | 2-8 | 0.07 | 0.12 | 0.06 | 0.06 | 0.12 | 0.3 |
| | 3 | 25 | 0.36 | 0.41 | 0.26 | 0.32 | 0.41 | 1.5 |
| Batch | 0 | | <0.1 | 0.07 | 0.03 | <0.1 | <0.1 | 0.1 |
| No. 5 | 3 | 2-8 | 0.10 | 0.15 | 0.07 | 0.08 | 0.15 | 0.4 |
| | 3 | 25 | 0.44 | 0.62 | 0.39 | 0.43 | 0.62 | 1.9 |
| Batch | 0 | | <0.1 | 0.13 | 0.07 | 0.04 | 0.13 | 0.2 |
| No. 6 | 3 | 2-8 | 0.07 | 0.17 | 0.11 | 0.03 | 0.17 | 0.4 |
| | 3 | 25 | 0.39 | 0.57 | 0.32 | 0.34 | 0.57 | 1.8 |

### Example 4

### Azithromycin Stability as a Function of Layered Storage Container

Five different samples of azithromycin were stored in a variety of packages to determine the amount of degradation products after a particular time and temperature. Using HPLC analytical methodology as described in Example 1, the presence and amount of degradation products for each package were determined. Each sample was packaged directly into an aluminum laminate, or packaged in an inner polyethylene (PE) bag and exterior aluminum laminate bag. Each sample was stored at an elevated temperature for 6-7 days. The results demonstrate that fewer azithromycin degradation products were found in the aluminum laminate bags as compared to the polyethylene/aluminum laminate double bag. Table 5 summarizes the effect of different packaging on the stability of azithromycin.

TABLE 5
Azithromycin Stability as a Function of Time

| | | | | RRT (%) | | | | | |
|---|---|---|---|---|---|---|---|---|---|
| AZT | | Time | | | | | | | Other |
| Batch | Package | (days) | T° C. | 0.25 | 0.33 | 0.36 | 0.78 | 0.80 | RRT % |
| Batch | | 0 | | <0.1 | 0.12 | <0.1 | <0.1 | <0.1 | 0.12 |
| No. 4 | direct in Al laminate | 6 | 55 | 0.17 | 0.14 | 0.07 | 0.11 | <0.1 | 0.17 |
| | PE bag in Al laminate | 6 | 55 | 0.49 | 0.48 | 0.26 | 0.35 | <0.1 | 0.49 |
| Batch | | 0 | | 0.09 | 0.08 | 0.03 | <0.1 | 0.06 | 0.10 |
| No. 5 | direct in Al laminate | 6 | 55 | 0.13 | 0.10 | 0.03 | 0.08 | 0.07 | 0.13 |
| | PE bag in Al laminate | 6 | 55 | 0.36 | 0.36 | 0.15 | 0.2 | 0.06 | 0.36 |
| Batch | | 0 | | 0.05 | 0.05 | 0.03 | <0.1 | <0.1 | <0.1 |
| No. 13 | direct in Al laminate | 6 | 55 | 0.14 | 0.12 | 0.05 | 0.05 | <0.1 | 0.14 |
| | PE bag in Al laminate | 6 | 55 | 0.42 | 0.44 | 0.19 | 0.27 | <0.1 | 0.44 |
| Batch | | 0 | | 0.37 | 0.38 | 0.19 | <0.1 | 0.22 | 0.38 |
| No. 7 | direct in Al laminate | 7 | 55 | 0.37 | 0.39 | 0.14 | <0.1 | 0.22 | 0.39 |
| | PE bag in Al laminate | 7 | 55 | 0.49 | 0.51 | 0.26 | <0.1 | 0.28 | 0.51 |
| Batch | | 0 | | 0.08 | 0.18 | 0.08 | <0.1 | <0.1 | 0.18 |
| No. 10 | direct in Al laminate | 7 | 55 | 0.12 | 0.25 | 0.10 | <0.1 | 0.06 | 0.25 |
| | PE bag in Al laminate | 7 | 55 | 0.24 | 0.41 | 0.18 | <0.1 | 0.15 | 0.41 |

### Example 5

### Double Aluminum Laminate Package Studies

Different batches of azithromycin were packaged in double aluminum laminate bags under a variety of conditions. The storage conditions included long term (2° C. to 8° C.); humid long term (25° C.±2° C. at 60%±5% relative humidity); humid accelerated (25° C.±2° C. at 60%±5% relative humidity); and high humidity accelerated (40° C. at 70%±5% relative humidity). After a predetermined amount of time, each sample was analyzed according to the analytical technique described in Example 1. Table 6 summarizes the test data. The decomposition of azithromycin in a double layer of aluminum laminate packaging was significantly inhibited. Even at a temperature of 40° C., the impurity increase was very moderate and close to the results at 25° C.

TABLE 6
Azithromycin Stability in Double Aluminum Bags.

| AZT | Time | RRT (%) | | | | Impurities | | % | % |
|---|---|---|---|---|---|---|---|---|---|
| Batch | (months) | 0.26 | 0.34 | 0.37 | 0.78 | Other RRT % | Total % | Water | EtOH |
| Batch No. | 0 | 0.29 | 0.40 | 0.17 | 0.12 | 0.40 | 1.30 | 3.22 | 2.1 |
| 10 | 3^{a} | 0.24 | 0.32 | 0.16 | 0.15 | 0.32 | 0.98 | 3.40 | 2.1 |
| | 3^{b} | 0.30 | 0.39 | 0.18 | 0.21 | 0.39 | 1.29 | 3.69 | 2.1 |
| | 1^{c} | 0.29 | 0.40 | 0.20 | 0.22 | 0.40 | 1.22 | 2.90 | 2.2 |
| | 2^{c} | 0.33 | 0.33 | 0.25 | 0.20 | 0.33 | 1.31 | 3.31 | 2.1 |
| | 3^{c} | 0.30 | 0.39 | 0.18 | 0.21 | 0.39 | 1.29 | 3.69 | 2.1 |
| | 1^{d} | 0.34 | 0.49 | 0.22 | 0.19 | 0.49 | 1.35 | 3.17 | 2.2 |
| | 2^{d} | 0.40 | 0.37 | 0.35 | 0.24 | 0.40 | 1.57 | 3.11 | 2.2 |
| | 3^{d} | 0.38 | 0.46 | 0.25 | 0.28 | 0.46 | 1.47 | 3.46 | 2.2 |
| Batch No. | 0 | <0.10 | 0.18 | 0.11 | <0.10 | 0.18 | 0.53 | 3.66 | 2.2 |
| 11 | 3^{a} | <0.10 | 0.15 | <0.10 | <0.10 | 0.15 | 0.26 | 3.90 | 2.1 |
| | 3^{b} | <0.10 | 0.19 | 0.12 | <0.10 | 0.19 | 0.54 | 3.75 | 2.1 |
| | 1^{c} | <0.10 | 0.20 | <0.10 | <0.10 | 0.20 | 0.37 | 3.69 | 2.1 |
| | 2^{c} | <0.10 | 0.16 | 0.12 | <0.03 | 0.16 | 0.41 | 3.77 | 2.1 |
| | 3^{c} | <0.10 | 0.19 | 0.12 | <0.10 | 0.19 | 0.54 | 3.75 | 2.1 |
| | 1^{d} | 0.12 | 0.24 | 0.12 | <0.10 | 0.24 | 0.75 | 3.65 | 2.1 |
| | 2^{d} | 0.15 | 0.18 | 0.18 | <0.10 | 0.18 | 0.65 | 3.47 | 2.2 |
| | 3^{d} | 0.21 | 0.31 | 0.15 | 0.11 | 0.31 | 0.90 | 3.84 | 2.1 |
| Batch No. | 0 | <0.03 | 0.13 | <0.10 | <0.03 | 0.16 | 0.42 | 3.67 | 2.2 |
| 12 | 3^{a} | <0.10 | <0.10 | <0.10 | <0.10 | 0.14 | 0.25 | 3.69 | 2.1 |
| | 3^{b} | <0.10 | 0.17 | <0.10 | <0.10 | 0.16 | 0.42 | 3.64 | 2.2 |
| | 1^{c} | <0.10 | 0.17 | <0.10 | <0.10 | 0.17 | 0.30 | 3.51 | 2.1 |
| | 2^{c} | <0.10 | 0.12 | 0.11 | <0.10 | 0.13 | 0.57 | 3.64 | 2.1 |
| | 3^{c} | <0.10 | 0.17 | <0.10 | <0.10 | 0.17 | 0.39 | 3.64 | 2.2 |
| | 1^{d} | 0.13 | 0.26 | <0.10 | <0.10 | 0.26 | 0.52 | 3.63 | 2.1 |
| | 2^{d} | 0.15 | 0.17 | 0.15 | <0.10 | 0.17 | 0.60 | 3.44 | 2.2 |
| | 3^{d} | 0.13 | 0.22 | <0.10 | <0.03 | 0.22 | 0.60 | 3.73 | 2.2 |

^{a}Long term.
^{b}Humid long term.
^{c}Humid accelerated.
^{d}High humidity accelerated.

### Example 6

### Year Long Azithromycin Study

Samples of azithromycin Form A were separately packaged into polyethylene/aluminum laminate bags, and each polyethylene/aluminum laminate bag was packaged into a second polyethylene/aluminum laminate bag. Each bag was subjected to a stability program (a) 25° C.±2° C. at 60% relative humidity or (b) 40° C.±2° C. at 75% relative humidity. After one year, each sample was analyzed as described in Example 1 to determine the presence and amount of degradation products. The impurity level for each sample was determined to be not more than 0.5%. Thus, each tested batch demonstrated the stability of azithromycin of greater than 1 year.

| Lot | Storage | | | | | | |
|---|---|---|---|---|---|---|---|
| No. | Conditions | Interval | RRT = 0.26 | RRT = 0.34 | RRT = 0.37 | RRT = 0.78 | Total |
| Lot 1 | 25° C./60% RH | 0 MT | <0.10 | 0.18 | 0.11 | <0.10 | 0.53 |
| | 25° C./60% RH | 1 MT | <0.10 | 0.20 | <0.10 | <0.10 | 0.37 |
| | 25° C./60% RH | 2 MT | <0.10 | 0.16 | <0.03 | <0.03 | 0.41 |
| | 25° C./60% RH | 3 MT | <0.10 | 0.19 | <0.10 | <0.10 | 0.54 |
| | 25° C./60% RH | 6 MT | 0.11 | 0.19 | <0.10 | <0.10 | 0.53 |
| | 25° C./60% RH | 9 MT | 0.13 | 0.19 | <0.10 | <0.10 | 0.60 |
| | 25° C./60% RH | 12 MT | 0.15 | <0.10 | <0.10 | <0.10 | 0.60 |
| | 25° C./60% RH | 18 MT | 0.17 | 0.19 | <0.10 | <0.10 | 0.91 |
| Lot 1 | 40° C./75% RH | 0 MT | <0.10 | 0.18 | 0.11 | <0.10 | 0.53 |
| | 40° C./75% RH | 1 MT | 0.12 | 0.24 | 0.12 | <0.10 | 0.75 |
| | 40° C./75% RH | 2 MT | 0.15 | 0.18 | 0.18 | <0.10 | 0.65 |
| | 40° C./75% RH | 3 MT | 0.21 | 0.31 | 0.15 | 0.11 | 0.90 |
| | 40° C./75% RH | 6 MT | 0.34 | 0.34 | 0.22 | 0.12 | 1.30 |
| Lot 2 | 25° C./60% RH | 0 MT | <0.03 | 0.13 | <0.10 | <0.03 | 0.42 |
| | 25° C./60% RH | 1 MT | <0.10 | 0.17 | <0.10 | <0.10 | 0.30 |
| | 25° C./60% RH | 2 MT | <0.10 | 0.12 | 0.11 | <0.10 | 0.57 |
| | 25° C./60% RH | 3 MT | <0.10 | 0.17 | <0.10 | <0.10 | 0.39 |
| | 25° C./60% RH | 6 MT | 0.1 | 0.15 | 0.10 | <0.10 | 0.46 |
| | 25° C./60% RH | 9 MT | 0.16 | 0.16 | 0.14 | <0.10 | 0.70 |
| | 25° C./60% RH | 12 MT | 0.18 | 0.25 | 0.16 | 0.11 | 1.00 |
| | 25° C./60% RH | 18 MT | 0.15 | 0.26 | <0.10 | 0.11 | 0.89 |
| Lot 2 | 40° C./75% RH | 0 MT | <0.03 | 0.13 | <0.10 | <0.03 | 0.42 |
| | 40° C./75% RH | 1 MT | 0.13 | 0.26 | <0.10 | <0.10 | 0.52 |
| | 40° C./75% RH | 2 MT | 0.15 | 0.17 | 0.15 | <0.10 | 0.60 |
| | 40° C./75% RH | 3 MT | 0.13 | 0.22 | <0.10 | <0.03 | 0.60 |
| | 40° C./75% RH | 6 MT | 0.16 | <0.10 | 0.12 | <0.10 | 0.56 |

The typical peak of azithromycin dihydrate in Form A is 13.2 degrees two-theta.

### Example 7

### Azithromycin Monohydrate Stability

A sample of azithromycin monohydrate is packaged into a polyethylene/aluminum laminate bag. The storage conditions include a temperature of about 25° C. and/or 60% relative humidity. After 3 months, the X-ray diffraction pattern shows that less than about 5% of azithromycin monohydrate is transformed to the dihydrate form.

What is claimed is:

1. A container for packaging azithromycin made of gas impermeable material wherein after storage azithromycin degradation products do not exceed 5% by weight of the azithromycin.

2. The container according to claim 1, wherein after storage azithromycin degradation products do not exceed 3% by weight of azithromycin.

3. The container according to claim 1, wherein the gas impermeable material is laminated aluminum.

4. The container according to claim 1 or 3, wherein the container comprises at least one additional layer of aluminum.

5. The container according to claim 1 or 2, wherein the azithromycin is azithromycin solvate.

6. The container according to claim 5, wherein the azithromycin is selected from the group consisting of ethanol solvate, propanol solvate, and a hydrate.

7. The container according to claim 6, wherein the azithromycin is monohydrate azithromycin.

8. A container for packaging azithromycin monohydrate made of gas impermeable material wherein after storage less than about 5% of azithromycin monohydrate is transformed to azithromycin dihydrate upon storage of one year.

9. The container according to claim 8, wherein the gas impermeable material is laminated aluminum.

10. The container according to claim 1, wherein the container is made of a bag or pouch.

11. The container according to claim 1, wherein the gas impermeable material is impermeable to oxygen.

12. The container according to any one of claims 1 and 2, wherein the gas impermeable is on the interior of the package.

13. The container according to any one of claims 1 and 2, wherein the azithromycin is stored at a temperature of about 25° C. to about 55° C.

14. The container according to any one of claims 1 and 2, wherein the azithromycin is stored at 60% relative humidity.

15. The container according to any one of claims 1 and 2, wherein the azithromycin is stored for at least one month.

16. The container according to any one of claims 1 and 2, wherein the degradation products are identified by HPLC relative to retention times of about 0.26, 0.34, 0.37, or 0.80.

17. A method for storing azithromycin comprising packaging azithromycin in a container comprising a gas impermeable material wherein after storage azithromycin degradation products do not exceed 5% by weight of the azithromycin.

18. The method according to claim 17, wherein the gas impermeable material is laminated aluminum.

19. The method according to claim 18, wherein the container further comprises at least one additional later of laminated aluminum.
